SEQUENCE LISTING (4 sequences)

SEQ ID NO: 1 - protein, 133 aa, Artificial Sequence
  Synthetic nanobody
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSHHHHHH

SEQ ID NO: 2 - protein, 133 aa, Artificial Sequence
  Synthetic nanobody
QVQLQESGGGLVCAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSHHHHHH

SEQ ID NO: 3 - protein, 127 aa, Artificial Sequence
  Synthetic nanobody
QVQLVESGGGLVCPGGSLTLSCAASGFSFSTTTMKWVRQAPGKGLERVSFINRDSTFTQYADSVKGRFTISRDNAKNTLYLQMSSLKPEDTAVYYCATASRITEGADFRGQGTQVTVSSGSHHHHHH

SEQ ID NO: 4 - protein, 133 aa, Artificial Sequence
  Synthetic nanobody
QVQLQESGGGLCQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSHHHHHH

(19) United States
(12) Patent Application Publication
(10) Pub. No.: US 2023/0258637 A1
(43) Pub. Date: Aug. 17, 2023

(54) METHODS FOR DETECTING A TARGET IN A SAMPLE USING MUTATED NANOBODIES

(71) Applicants: UNIVERSITE DE LILLE, (US); CENTRALE LILLE INSTITUT, (US); UNIVERSITE POLYTECHNIQUE HAUTS-DE-FRANCE, (US); CENTRE NATIONAL DE LA RECHERCHE SCIENTIFIQUE - CNRS -, (US); INSERM (INSTITUT NATIONAL DE LA SANTÉ ET DE LA RECHERCHE MÉDICALE), (US); CENTRE HOSPITALIER UNIVERSITAIRE DE LILLE, (US); UNIVERSITÉ D'AIX-MARSEILLE, (US)

(72) Inventors: Sabine SZUNERITS, Villeneuve d'Ascq (FR); Alain ROUSSEL, Marseille (FR); Christian CAMBILLAU, Roma (IT); David DEVOS, Marcq-en-Baroeul (FR); Ilka ENGELMAN, Emmerin (FR); Enagnon Kazali ALIDJINOU, Roubaix (FR)

(21) Appl. No.: 18/031,242

(22) PCT Filed: Oct. 12, 2021
(86) PCT No.: PCT/EP2021/078184

§ 371 Date: Apr. 11, 2023

(30) Foreign Application Priority Data
Oct. 12, 2020 (EP) 20306199.9

(51) Int. Cl. G01N 33/569; G01N 33/543; C07K 16/10

(52) U.S. Cl. CPC G01N 33/56983; G01N 33/5438; C07K 16/10; G01N 2333/165; C07K 2317/569; C07K 2317/567

(57) ABSTRACT

The present invention relates to methods for detecting a target in a sample using mutated nanobodies, wherein an amino acid present in the loop of the FR1 region of framework of the nanobodies is mutated to cysteine.

### TECHNICAL FIELD

The present disclosure relates to methods for detecting a target in a sample using mutated nanobodies.

### BACKGROUND

Epidemic and pan epidemic outbreaks, as the one the world is facing currently with the COVID-19 outbreak, can be better managed with the availability of efficient, portable and cheap diagnostic devices.

The market of “viral biosensors” is still in its infancy and cannot respond to the current demands of screening a large part of the population being infected, neither providing a tool where daily diagnostic can be achieved in an easy and personalized manner. Biosensors hold however great impact to turn current analytical methods into diagnostic strategies by simple restructuring their sensing module for the detection of protein biomarkers and viruses (Dong et al., Bioelectrochemical and Bioenergetics. 1997, 42, 7; Xue et al., Nature Communications. 2014, 5, 4348; Yang et al., Journal of Electroanalytical Chemistry 2001, 516, 10).

Unquestionably, such sensing platforms require continuous updates to address growing challenges in the diagnosis of viruses as these are changed quickly and spread largely from person-to-person, indicating the urgency of early diagnosis. This requires the development of an easily sensing platform, which can be adapted quickly to changing demands. Furthermore, with the rise in personalized medicine and adapted technologies for wireless sensing, current as well as future viral contaminations of millions of people can be controlled and limited. The requirement for weekly confinement can in this manner be limited and even avoided, with unimaginable positive consequences for the economy of a country and the globe.

Currently, the reference diagnosis are real-time reverse transcriptase PCR assays on nasal swabs (effective in early infection with signs of nasopharyngeal involvement) and on tracheal swabs (effective in the event of infection at a slightly later stage with broncho-pneumopathy but more invasive and therefore reserved for severe cases). The time required to obtain RT-PCR results is long, which considerably limits the rapid management measures necessary in the event of a pandemic. In addition, there is the question of mass screening, particularly of asymptomatic patients. One of the major challenges is to gain time in analysis, but also to be highly specific and decrease the amount of false negative responses, as often occurring in RT-PRC analysis.

The technical solution to this problem could therefore be the use of an intelligent portable biosensor based electrochemical read out through the surface immobilization of nanobodies specific to a target of interest for the fast and selective sensing of such target.

Over the years, the possibility of detecting a target substance through antibody immobilization onto a surface, also called, immunosensor or immunobiosensors, has been documented. However, batch-to-batch inconsistency of polyclonal antibodies and extensive production time of monoclonal antibodies, pose additional limitations.

Single domain antibodies, also called camelid-derived antibodies or nanobodies, are the recombinantly expressed binding fragments derived from heavy chain antibodies found in camels and llamas. These unique binding elements offer many desirable properties such as their small size (˜15 kDa) and thermal stability, which makes them attractive alternatives to conventional monoclonal antibodies. These nanobodies have been reported to be inherently unaffected by changes in temperature and retain their structure due to high refolding efficiency (Ingram et al., Exploiting Nanobodies' Singular Traits, 2018; Goldman et al., Enhancing Stability of Camelid and Shark Single Domain Antibodies, 2017; Goldman et al., Analytical Chemistry 2006, 78, 8245). Successful utilization of these nanobodies in sandwich immunoassays immobilized on magnetic microspheres, for example, has been demonstrated effective in detecting various pathogens and toxins (Anderson et al., Analytical Chemistry 2008, 80, 9604; Anderson et al., Analytical Chemistry 2010, 82, 7202.

US2017059561A1 describes single-domain antibodies as the sensing agents immobilized onto a surface of an immunosensing device. However, to facilitate the immobilization of the nanobody, the conductive electrode was chemically functionalized by at least one self-assembled monolayer prior to the immobilization of the nanobody which is time consuming. Further, the orientation of the nanobody to optimize its target recognition capability is not discussed.

The use of nanobodies for the development of materials for specific portable biosensors for rapid reading, for example, has never been reported. There is therefore an unfulfilled need for a portable detection device that is easy to design, cheap to produce while being specific to a target of interest, having a fast reading and possessing good statistic results.

The inventors of the present invention have discovered that the mutation of a nanobody in a specific region of its sequence makes it possible to graft it directly onto a surface while making it possible to correctly orient the paratope of the nanobody. The resulting sensing platform thus makes it possible to solve the aforementioned technical problems by allowing rapid detection at low costs, while being specific to a target of interest.

### SUMMARY

The present inventors have found a way to design and produce a “biosensor” based on an electrochemical read out though the surface immobilization of mutated nanobodies specific to a target, for example the SARS-CoV-2 receptor binding domain (RBD) protein.

One first aspect of the invention relates to a mutated nanobody which binds to a target wherein an amino acid present in the loop of the FR1 region of the framework, preferably at position 12, 13, 14 or 15, is mutated to cysteine.

In another aspect, the invention relates to the mutated nanobody of the present invention, wherein the mutated nanobody is attached to a surface.

The present invention further relates to a biosensor comprising the surface of the present invention, for detecting a target in a sample.

Finally, the present invention further relates to a method for detecting a target in a sample, the method comprising:

a. Providing the biosensor of the present invention,

b. Contacting the biosensor with the sample,

c. Measuring the response at the biosensor surface, and

d. Determining the presence or the absence of the target.

### DETAILED DESCRIPTION OF THE INVENTION

### Definitions

Terms as used throughout the invention are hereafter defined.

As used herein, the term “target” refers to a sensing target that is selected from the group consisting of viral proteins, protein biomarkers, bacteria proteins, membrane proteins, protozoa proteins, fungi proteins, and prion proteins. In an embodiment, the target originates from infectious agents. In a preferred embodiment, the target is a viral protein, preferably a SARS virus protein, more preferably SARS-CoV-2 virus protein, and even more preferably SARS-CoV-2 receptor binding protein (RBP). In another preferred embodiment the target is a HSV-1 virus protein. Additional examples of infectious agents are well known to a person of ordinary skill in the art and such embodiments are within the purview of the current invention.

As used herein, the term “sample” refers to biofluids such as nasal swab, mouth swab, spit, blood, amniotic fluid, aqueous humor, vitreous humor, bile, cerebrospinal fluid, chyle, endolymph, perilymph, female ejaculate, male ejaculate, lymph, mucus (including nasal drainage and phlegm), pericardial fluid, peritoneal fluid, pleural fluid, pus, rheum, sputum, synovial fluid, vaginal secretion. In a preferred embodiment, the sample of the present invention is selected from the group consisting of nasal or mouth swab, spit, and blood. The biofluid can be processed prior to analysis according to the current invention. For example, blood can be processed to make it suitable for analysis by the electrochemical biosensor of the current invention, for example, by centrifugation to removal of the cells or by deproteinization. The person skilled in the art knows how to process the sample depending on its origin, in order to make it suitable for analysis by the biosensor device of the present invention.

If not stated herein otherwise, “about” means±20%, preferably ±10%, more preferably ±5%.

The Mutated Nanobody

Nanobodies are a class of antigen-binding protein derived from camelids comprising only a single 15 kDa variable domain. They exhibit improved stability and are able to bind a large number of epitopes, sometimes even to those not accessible to classical antibodies.

In the present invention we rely on the nomenclature described in the article Laura S. Mitchell, Lucy J. Colwell “Comparative analysis of nanobody sequence and structure data” Proteins. 2018; 1-10 which is hereby incorporated by reference. This article describes the structure of nanobodies, as shown in FIG. 1, which contain three highly variable loops CDR1, CDR2, and CDR3 that also may be called H1, H2 and H3. These loops form an extended structural interface at one side of the folded protein domain that contributes to the antigen-binding interface, or paratope, which determines the nanobody antigen-binding specificity.

Between these three CDR regions there is the framework. This framework is composed of 4 regions called FR1, FR2, FR3 and FR4. Laura S. Mitchell and Lucy J. Colwell article has studied the frequency of mutation from one nanobody to another which is shown in FIG. 2.

As demonstrated, nanobodies do not diversify their framework region from one to another to compensate for the loss of the VL domain. Based on the nomenclature of Laura S. Mitchell and Lucy J. Colwell article, the framework from one nanobody to another extends from position 1 to position 25 for FR1, from position 36 to position 49 for FR2, from position 60 to position 97 for FR3 and from position 117 to position 126 for FR4.

The mutated nanobody of the invention is mutated in the framework of the nanobody. To determine the positions of the amino acids in the sequence and to determine the position of the mutated amino acid, we will use in the present invention the nomenclature of Laura S. Mitchell and Lucy J. Colwell article. This framework being conserved from one nanobody to another, the mutation is applicable to all nanobodies. According to the nomenclature of Laura S. Mitchell and Lucy J. Colwell article, the mutation is made in positions which are in the loop of the FR1 region and the mutation consists in replacing the amino acid present at the aforementioned position by a cysteine (Cys or C). This mutation is made at a position that is at the opposite side relative to the paratope, and thus, from the highly variable CDR regions of the nanobody. Indeed, being in such position from the paratope allows to use the mutated nanobody of the present invention to be grafted on a surface in such a way as to orientate the paratope in the opposite direction to the surface while avoiding dimerization. Preferably, the mutation is made in the FR1 region at position 12, 13, 14, or 15.

One first aspect of the invention relates to a mutated nanobody which binds to a target wherein an amino acid present in the loop of the FR1 region, preferably at position 12, 13, 14, or 15, more preferably at position 13 is mutated to cysteine.

Nanobodies are well-known for the person skilled in the art (Serge Muyldermans “Nanobodies: Natural Single-Domain Antibodies” Annu. Rev. Biochem. 2013. 82:775-97; Laura S. Mitchell, Lucy J. Colwell “Comparative analysis of nanobody sequence and structure data” Proteins. 2018; 1-10). Therefore, according to the target to be detected, the person skilled in the art will know how to produce the nanobody specific to said target and thus to transpose the aforementioned technology according to the present invention to any nanobody specific to any target. The process of nanobody generation through llama immunization and phage display is described in Desmyter et al, Curr Opin Struct Biol. 2015; 32:1-8. The present invention is therefore not limited but encompasses all the possibilities of detection of a target of interest by a specific nanobody that is mutated according to the present invention.

Typically, the mutated nanobody of the present invention may comprise a tag sequence, such as for example a polyhistidine tag. Typically, the mutated nanobody of the present invention may be linked to another peptide or polypeptide, such as for example to another nanobody to form diabodies. The other nanobody may bind to the same target as the target of the mutated nanobody of the present invention or to an alternative target.

The Mutated Nanobody Attached to a Surface

Nanobodies have been successfully applied for the development of biosensors. Biosensors are devices where a bioreceptor, in this case a nanobody, is in close contact with a surface, in particular a transducer, that converts the biorecognition event into a measurable signal.

In an embodiment of the present invention, the mutated nanobody is attached to a surface. By “surface” it is referred to in the present invention a conductive substrate that may be flat or curved. In an embodiment, the surface is a gold surface and preferably a working electrode. In another embodiment, the surface is selected from the group consisting of graphene, reduced graphene oxide and its derivatives, or a metal surface other than gold such as carbon, platinum, nickel, copper, silver. In a preferred embodiment, the surface is graphene surface. Preferably, the surface is a working electrode. In a preferred embodiment, the surface is the working electrode of a biosensor device.

The present invention also relates to a biosensor comprising the surface of the present invention for detecting a target in a sample.

The nanobody modified working electrode became a biosensor capable of analyzing in real time intermolecular interactions on a sensor chip with the use of electrochemical techniques respectively. A gold surface layer or a surface layer selected from the group consisting of graphene, reduced graphene oxide and its derivatives, or a metal surface other than gold is formed on a sensor chip, so that the cysteine-modified nanobody of the present invention can be grafted stably with its high binding capability. If the nanobody interacts with the target in the sample, binding and dissociation can be detected in real time.

Regarding the electrochemical sensor, the changes in the electrochemical properties, for example, electron transfer properties, at the sensing electrode surface upon binding of the target to the nanobody attached to the sensing electrode surface can be measured by a test equipment.

Various electrochemical properties at the sensing electrode surface that can be measured by the test equipment include, but are not limited to, electrical resistance, potential and current changes. In certain embodiments, the test equipment, is a voltmeter, ohmmeter, ammeter, multimeter, or a potentiostat. In a further embodiment, the tests performed with this equipment may be a differential pulse voltammetry, electrochemical impedance spectroscopy (EIS), cyclic voltammetry (CV), linear sweep voltammetry (LSV) chronoamperometry (CA), or chronopotentiometry.

Differential pulse voltammetry (DPV) is a voltammetry method used to make electrochemical measurements. It is a derivative of linear sweep voltammetry, with a series of regular voltage pulses superimposed on the potential linear sweep or stairsteps. The current is measured immediately before each potential change, and the current difference is plotted as a function of potential. By sampling the current just before the potential is changed, the effect of the charging current can be decreased in favor of the faradic current making the method highly sensitive.

DPV allows direct analyses down to pM protein concentrations and low bacteria concentrations among others. In the present invention, the redox probe used is ferrocene-methanol (FcMeOH) but can be any other redox couple such as ferroceylamine (Fc-NH₂), ferricyanide (Fe(CN)₆³⁻] ferrocyanid Fe(CN)₆⁴⁻], ruthenium hexamine (Ru(NH₃)₆), etc. for the detection of interaction of the target with the grafted nanobody as interaction will considerably decrease the electron charge transfer interaction. The presence of the target acts as a diffusion barrier, hindering the charge transfer from the redox probe to the surface. Addition of the target present in a sample results in a decrease of the redox current as the complex nanobody-target forms a barrier.

Therefore, electrochemical sensor to which the nanobody of the present invention has been bound is useful as a biosensor device for analysis of target-nanobody interactions.

In a further aspect, the present invention relates to a method for detecting a target, preferably a virus, in a sample, the method comprising:
a. Providing the biosensor as defined in the present invention,
b. Contacting the biosensor with the sample,
c. Measuring the electrochemical response at the bio sensor surface, and
d. Determining the presence or the absence of the target.

In the method of the current invention the sample containing the target to be measured is contacted with the sensor under conditions that allow the formation of the nanobody-target complexes for a period of time sufficient to allow the formation such complexes. In a preferred embodiment, the sample containing the target to be measured is contacted with the sensor under conditions that allow or promote only specific binding between the paratope of the nanobody of the present invention and the target in the sample. The nanobody of the present invention being specific to the target of interest, non-specific binding between the nanobody and other chemicals in the sample is avoided. A period of time sufficient to allow the formation of the nanobody-target complexes can be about 1 minute to about 60 minutes, about 1 minutes to about 10 minutes, or about 5 minutes.

Grafting of the Nanobody of the Present Invention onto a Surface

The nanobody of the present invention can be attached to the surface in various ways. In certain embodiments, the nanobody is attached to the surface in a covalent manner. Binding in a covalent manner ensures that the nanobody is, for practical purposes, permanently attached to the sensor which avoids the loss of the nanobody, for example, during the washing of the sensor. In another embodiment, the nanobody is attached to the surface in a non-covalent manner.

The immobilization of nanobodies onto the surface is a pivotal step for the construction of the biosensor device of the present invention, as it affects selectively and sensitivity. In an embodiment, the sensor modification is based on the interaction of thiolate groups of the nanobody with gold through the formation of Au—S bond, classically used for gold electrode modification. The strength of the gold-sulphur (Au—S) interaction formed between thiols and gold surfaces provides the basis to fabricate robust self-assembled monolayers for diverse applications. In another embodiment, the sensor modification is based on the interaction of the surface selected from the group consisting of graphene, reduced graphene oxide and its derivatives, or a metal surface other than gold such as carbon, platinum, nickel, copper, silver, with the thiolate groups of the nanobody which provides the basis to fabricate robust self-assembled monolayers for diverse applications.

In certain aspects, the sensor modification is based on the interaction of a short linker between the nanobody and gold surface or a surface selected from the group consisting of graphene, reduced graphene oxide and its derivatives, or a metal surface other than gold such as carbon, platinum, nickel, copper, silver, preferably gold or graphene, in such a way that the cysteine of the nanobody is at a distance of less than 1 nanometer (1 nm) of said surface. The short distance allows to fabricate robust self-assembled monolayers for diverse applications. In certain aspects, the short linker may be a pyrene-based linker with attached functional ending such as COOH, NH₂, or maleimide (MAL), or PEG-aryl radicals such as PEG-aryldiazonium salts, that attacks the surface through the reduction of aryldiazonium ions. Examples of aryldiazoniums salts are 4-[(triisopropylsilyl)ethylenyl]benzenediazonium tetrafluoroborate (TIPS-Eth-ArN2+), 4-nitrobenzene diazonium tetrafluoroborate, 4-Methoxybenzenediazonium tetrafluoroborate, 4-Bromobenzenediazonium tetrafluoroborate, and 4-carboxylicacid benzenediazonium tetrafluoroborate. In an embodiment, the functional groups of the aryldiazonium attached to the gold surface or a surface selected from the group consisting of graphene, reduced graphene oxide and its derivatives, or a metal surface other than gold such as carbon, platinum, nickel, copper, silver may be post-functionalized with PEG-maleimide, PEG-iodoacetyl1-pyrenebutyric acid (PBA), 1-pyrenebutanoic acid succinimidyl ester (PBASE), or pyrene-maleimide (Py-MAL), which is linked to the thiolate groups of the nanobody.

Nanobodies of the present invention are mutated in order to replace an amino acid of the framework by a cysteine. The sulfide group of the cysteine residue allows to graft directly the mutated nanobody of the present invention onto the gold surface. Thanks to the remote position of the cysteine residue inserted in the sequence with respect to the paratope, the nanobody of the present invention is oriented in such a way that the paratope in the opposite direction to the surface of the sensor. In this way, each nanobody grafted onto the surface is capable of binding the target present in the sample. Further, the direct grafting of the nanobody onto the surface or the grafting through a short linker of the present invention allows to reduce the distance that the electrons have to travel in order to trigger the biosensor signal. In such a way, the mutated nanobody of the present invention allows to be grafted easier, rapidly and to provide a biosensor which is specific to a target of interest, having a fast reading. In an embodiment, the grafting does not use a linker between the nanobody and the surface of the sensor.

In an embodiment, the amount of the mutated nanobody used for the surface grafting is between 0.01 and 20 mg/mL, preferably between 0.05 and 10 mg/mL, and more preferably between 0.1-1 mg/mL. The time for grafting is between 2 and 24 h preferably between 3 and 12 h and more preferable between 9 and 12 h. The solution pH of the PBS 1× dilution is between 5 and 8, preferable between 6 and 8 and more preferable between 7.4 and 8.

### LISTING OF FIGURES

FIG. 1: This figure corresponds to FIG. 1 (B) from the article Laura S. Mitchell, Lucy J. Colwell “Comparative analysis of nanobody sequence and structure data” Proteins. 2018; 1-10 showing Left: the secondary structure of the nanobody VHH domain which consists of 9 beta sheets separated by loop regions, 3 of which are hypervariable (H1, H2 and H3). Four framework regions (FRs) separate the variable loops.

FIG. 2: This figure corresponds to FIG. 3 (A) from the article Laura S. Mitchell, Lucy J. Colwell “Comparative analysis of nanobody sequence and structure data” Proteins. 2018; 1-10 showing sequence logo plots that show the amino acid variation between nanobodies.

FIG. 3: Differential pulse voltammograms of grafted gold electrodes of example 2 VHH C13 (A) and VHH C12 (B). Solution Ferrocenmethanol (FcMeOH, 1 mM) in PBS (0.1M). DPV Parameters: equilibrium time 3s; Estep=0.01V; Epuls=0.06V; T=0.02s; scan rate: 0.06V/s.

FIG. 4: Change of current as a function of the log concentration of the receptor binding protein: Calibration curve of differential pulse voltammograms of grafted gold electrodes of example 2 VHH C13 according to different concentration of the receptor binding protein of SARS-CoV-2. Solution Ferrocenmethanol (FcMeOH, 1 mM) in PBS (0.1M). DPV Parameters: equilibrium time 3s; Estep=0.01V; Epuls=0.06V; T=0.02s; scan rate: 0.06V/s.

FIG. 5: Dilution testing of grafted gold electrodes of example 2 compared to RT-PCR. White column corresponds to RT-PCR, light grey corresponds to VHH 72 biotin, dark grey corresponds to VHH C13 of the present invention and black corresponds to non-mutated VHH 72.

FIG. 6: Results of three different sensing interfaces modified with non-mutated VHH 72, biotin-modified VHH 72 and cystein modified nanobody VHH C13 using samples from patients where RT-PCR gave Covid-19 positive or negative results. In the case of VHH C13 results concordance between RT-PCR and the mutated SARS-CoV-2 nanobody of the present invention is made on the basis of 100 PCR negative samples (dark grey bullets) and 73 PCR positive samples (light grey bullets).

FIG. 7: Change of current as a function of the log of the concentration of HSV-1 virus.

FIG. 8: Change of current as a function of the log concentration cultured SARS-CoV-2 viral particles of example 2 VHH C12 (A) and VHH C13 (B). Solution Ferrocenmethanol (FcMeOH, 1 mM) in PBS (0.1M). DPV Parameters: equilibrium time 3s; Estep=0.01V; Epuls=0.06V; T=0.02s; scan rate: 0.06V/s

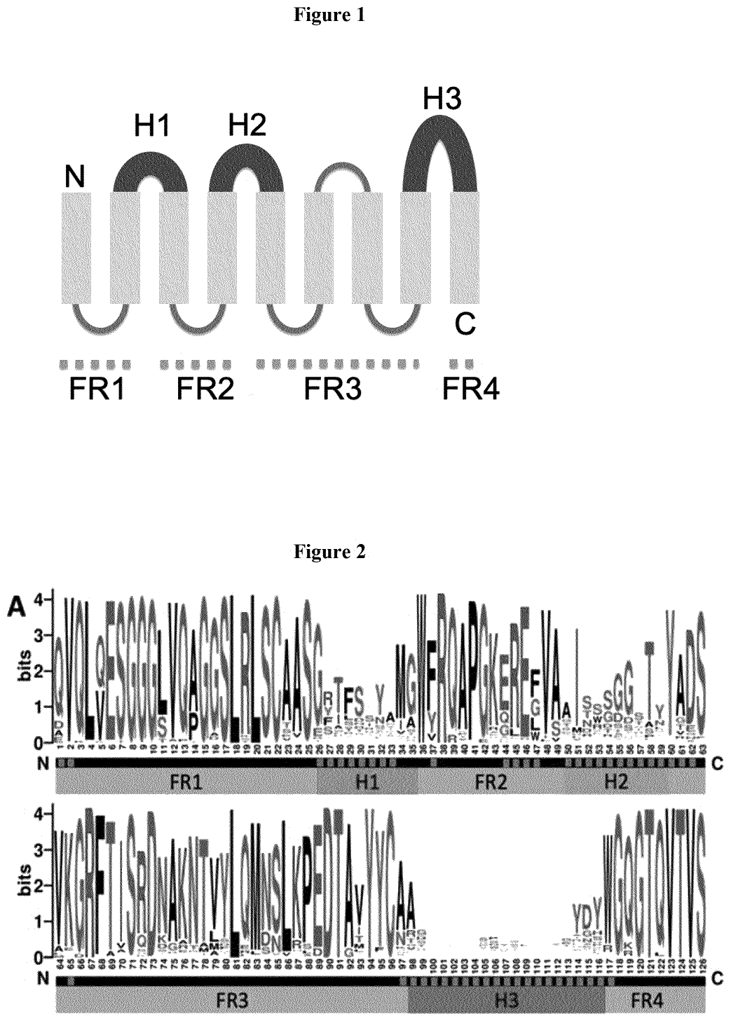

### EXAMPLES

### Example 1: Production of Non-Mutated and Mutated Nanobody Specific to the Receptor Binding Protein of the SARS-Cov-2 Virus

1. Production of the Non-Mutated Nanobody Specific to the Receptor Binding Protein of the SARS-Cov-2 Virus: VHH 72

The sequence of the non-mutated nanobody specific to the receptor binding protein of the SARS-Cov-2 virus is as follows:

(SEQ ID NO: 1)
QVQLQESGGGLVQAGGSLRLSCAASGRTFSEYAMGWFRQAPGKEREFVA
TISWSGGSTYYTDSVKGRFTISRDNAKNTVYLQMNSLKPDDTAVYYCAA
AGLGTVVSEWDYDYDYWGQGTQVTVSSGSHHHHHH

The nanobody contains a C-terminal polyhistidine tag in order to facilitate the purification.

This non-mutated nanobody was produced in T7 Express Escherichia coli cells (NEB) cultured in Turbo Broth medium (Athena) at 37° C. for 4 h. At this stage, the expression was induced with 0.3 mM IPTG and the temperature was decreased to 17° C. and the cells were grown for an additional 18 h. Cells were pelleted by centrifugation for 10 min, 5000 g, at 4° C. The pellets were flash freezed in liquid nitrogen then thawed at room temperature. The pellets were then resuspended in lysis buffer (50 mM Tris, 300 mM NaCl, 5% glycerol, 0.1% Triton, 5 mM Imidazole, 20 ug/ml DNase, 0.1M PMSF, 0.1 mg/ml lysozyme) and put under agitation for 45 minutes at 4° C. The cells were sonicated at 50% amplitude for 3 rounds of 30 seconds. The lysat was centrifugated at 13000 g for 30 minutes at 4° C. and the supernatant was then purified on a 5 ml Ni-NTA column (GE Healthcare) in 50 mM Tris, 5% glycerol, 5 mM Imidazole, 300 mM NaCl, pH 8.0. The fractions eluted in 250 mM imidazole were concentrated by centrifugation using an Amicon Ultra 10 kDa cutoff concentrator prior to being loaded onto a HiLoad 16/60 Superdex 75 pg gel filtration column (GE Healthcare) equilibrated in phosphate buffered saline (PBS). The purified nanobodies were concentrated by centrifugation; their concentration was determined by measuring the absorbance at 280 nm with a NanoDrop 2000 (Thermo Scientific).

2. Production of the Mutated Nanobody Specific to the Receptor Binding Protein of the SARS-Cov-2 Virus of the Present Invention VHH C13

The non-mutated nanobody of point 1 was mutated at position 13 in order to replace glutamine (Q) by a cysteine (C) following the procedure hereafter. A synthetic gene encoding the mutated protein has been ordered from Twist bioscience. The mutated nanobody was produced following the same protocol as described in point 1 for non-mutated nanobody.

The sequence obtained of the mutated nanobody of the present invention VHH C13 is as follows:

(SEQ ID NO: 2)
QVQLQESGGGLVCAGGSLRLSCAASGRTFSEYAMGWFRQAPGKEREFVA
TISWSGGSTYYTDSVKGRFTISRDNAKNTVYLQMNSLKPDDTAVYYCAA
AGLGTVVSEWDYDYDYWGQGTQVTVSSGSHHHHHH

3. Production of the Mutated Nanobody Specific to the Receptor Binding Protein of the SARS-Cov-2 Virus VHH-Biotin

The non-mutated nanobody of point 1. was labelled with biotin following the procedure hereafter. 1 mg of the non-mutated nanobody at 4 mg/mL in 0.1M bicarbonate buffer pH 9 (100 mL:80 mL H20+0.765 g Na Bicarbonate+0.095 Na carbonate, adjust at 100 mL with H20) was mixed with dye dissolved in DMSO at 10 mg/mL. The mixture was kept for 1 hour under permanent stirring away from light before centrifugation and gel filtration.

4. Production of the Mutated Nanobody Specific to the Receptor Binding Protein of the SARS-Cov-2 Virus of the Present Invention VHH C12

The non-mutated nanobody of point 1 was mutated at position 12 in order to replace valine (V) by a cysteine (C) following the procedure hereafter. A synthetic gene encoding the mutated protein has been ordered from Twist bioscience. The synthetic was inserted in a vector for mammal expression. The mutated nanobodies fused with a FC domain were produced in HEK Expi293 cells cultured in Expi293 expression medium from ThermoFisher at 37° C., 150 rpm until the cells were around 1.10\hat{ }6 cells/mL. At this stage, cells were transfected with 75 μg of DNA and 225 μg of PEI Max-transfection grade linear (Polysciences) and the cells were grown for an additional 96h. After the first 24h, additives were added on the cells: 0.5 mM of valproic acid, 4 g/L of glucose and 20% tryptone N1. After 96h, cells were pelleted by centrifugation for 10 min, 700 g, at 4° C. and the supernatant was then purified on a 5 ml Ni-NTA column (GE Healthcare) in 50 mM Tris, 5% glycerol, 5 mM Imidazole, 300 mM NaCl, pH 8.0. The fractions eluted in 250 mM imidazole were concentrated by centrifugation using an Amicon Ultra 10 kDa cutoff concentrator prior to being loaded onto a HiLoad 16/60 Superdex 75 pg gel filtration column (GE Healthcare) equilibrated in phosphate buffered saline (PBS). The purified nanobodies-FC were concentrated by centrifugation; their concentration was determined by measuring the absorbance at 280 nm with a NanoDrop 2000 (Thermo Scientific). The VHH and the FC domain were separated by the use of TEV protease with 1:10 ratio. After one night of cleavage at room temperature, the product was loaded onto a HiLoad 16/60 Superdex 75 pg gel filtration column equilibrated in PBS. The purified nanobodies were concentrated by centrifugation using an Amicon Ultra 3 kDa cutoff concentrator, the concentration was determined by measuring the absorbance at 280 nm and the denaturation curve was determined using a Tycho NT.6 from Nanotemper Technologies.

The sequence obtained of the mutated nanobody of the present invention VHH C12 is as follows:

(SEQ ID NO: 4)
QVQLQESGGGLCQAGGSLRLSCAASGRTFSEYAMGWFRQAPGKEREFVA
TISWSGGSTYYTDSVKGRFTISRDNAKNTVYLQMNSLKPDDTAVYYCAA
AGLGTVVSEWDYDYDYWGQGTQVTVSSGSHHHHHH

### Example 2: SARS-CoV-2 Nanobodies Grafting on Gold Electrodes

Before the grafting of SARS-CoV-2 nanobodies of example 1 on gold electrodes, the gold interfaces were cleaned following the procedure hereafter:

Electrodes were irradiated with UV-OZONE for 5 min. Then washed with mQ water dried with a dry air. In the second time, electrodes were cleaned electrochemically with 0.5M H₂SO₄ solution. For this, connect the electrodes to the potentiostat (Palmsens, Sensit), deposit H₂SO₄ on the electrodes and start cleaning method (Table.1). When the method is finished, rinse electrodes with mQ water and then dry with a dry air.

TABLE 1
Cleaning parameters

| Parameter | Value |
|---|---|
| Technique | Cycle Voltammetry |
| Current range | Full Range (100 pA-100 mA) |
| Pretreatement settings | All parameters at 0 |

Cyclic Voltammetry

| | | |
|---|---|---|
| T equilibration | 5 | s |
| E begin | −0.2 | V |
| E vertex 1 | 1 | V |
| E vertex 2 | −0.2 | V |
| E step | 0.001 | V |
| Scan Rate | 0.1 | V/s |

| | |
|---|---|
| Number of scans | 10 |

Once cleaned, 3 types of gold electrodes were produced using the 3 different nanobodies of example 1.

1. Gold Electrode Grafting with the Mutated Nanobody of the Present Invention: VHH C13 or VHHC12

The gold electrode is prepared in two steps: The Au electrode is exposed to 10 μL of an aqueous solution of 3-mercaptoproponic acid (25 mM) for 30 min at room temperature. Then acid-terminated surface is activated with EDC/NHS (1:1 molar ratio, 15 mM) for 20 min, followed by immersion into NH₂-PEG₆-maleimide (10 μL, 0.1 mg/m, in PBS 1×) for 2 h at 4° C. and washed with MQ-water. A solution at 100 μg/mL in PBS 1× of SARS-CoV-2 nanobody VHH C13 of example 1 point 2 or VHHC12 of example 1 point 4 is dropped on electrodes and keeping a 4° C. overnight under humid atmosphere. Surfaces were wash, dry and keep it a 4° C. until use.

2. Gold Electrode Grafting with the Non-Mutated Nanobody VHH

A solution L-Cysteine (SigmaAldrich, France) at 2 mM on PBS 1× were dropped on electrodes and keeping at 4° C. overnight under humid atmosphere. Electrodes were washed, dried and a solution of NHS/EDC (SigmaAldrich, France) at 15 mM was dropped on surfaces (2h at 4° C.) after 2 hours electrodes were washed, dried and a solution at 100 μg/mL of SARS-Cov-2 nanobody VHH of example 1 point 1 was incubated overnight at ° 4° C. under humid atmosphere. Surfaces were wash, dry and keep it a 4° C. until use.

3. Gold Electrode Grafting with the Biotin Mutated Nanobody: VHH-Biotin

A solution L-Cysteine at 2 mM on PBS 1× were dropped on electrodes and keeping at 4° C. overnight under humid atmosphere. Electrodes were washed, dried and the surfaces were incubated 2 hours at 4° C. with NHS/EDC at 15 mM. After 2 hours electrodes were washed, dried and a solution of streptavidine (ThermoFisher, France) was incubated with the surfaces at ° 4° C. under humid atmosphere overnight. Next day, electrodes were washed, dried and a solution of SARS-Cov-2 nanobody VHH-biotin of example 1 point 3 at 100 μg/mL is incubated for 2 hours. Surfaces were wash, dry and keep it a 4° C. until use.

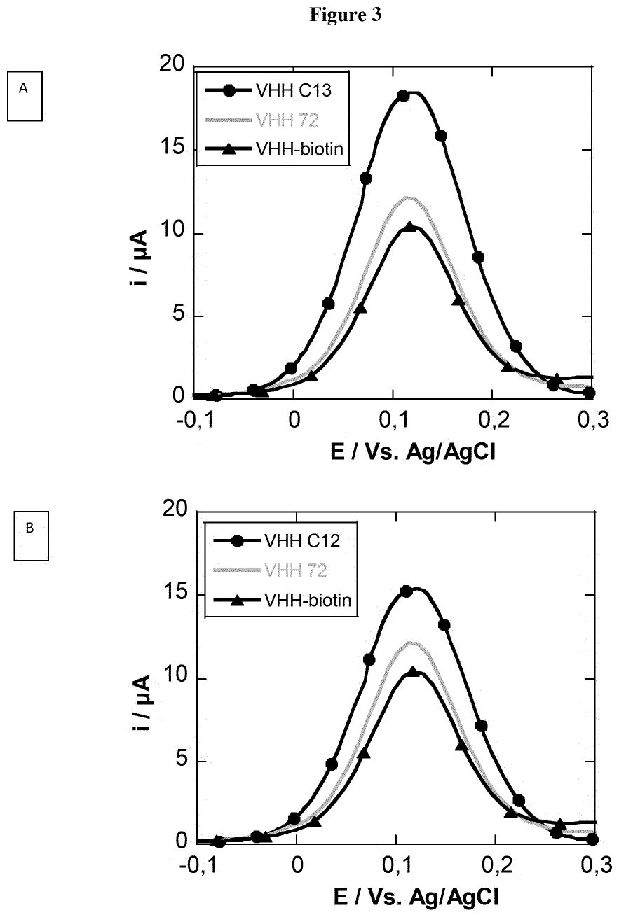

After the grafting of each gold electrode, differential pulse voltammograms (DPV) were measured for each final electrode. The solution used of the measure is Ferrocenmethanol (FcMeOH, 1 mM) in PBS (0.1M). DPV Parameters are as follows: equilibrium time 3s; Estep=0.01V; Epuls=0.06V; t=0.02s; scan rate: 0.06V/s. The result of the DPV is in FIG. 3. As can be seen, the gold electrode grafting with the mutated nanobody of the present invention VHH C13 (FIG. 3A) and VHH C12 (FIG. 3B) allows having a large current notably by the fact that the mutated nanobody of the present invention blocks less the transfer of electrons from the nanobody to the electrode. This is not the case with the other electrodes.

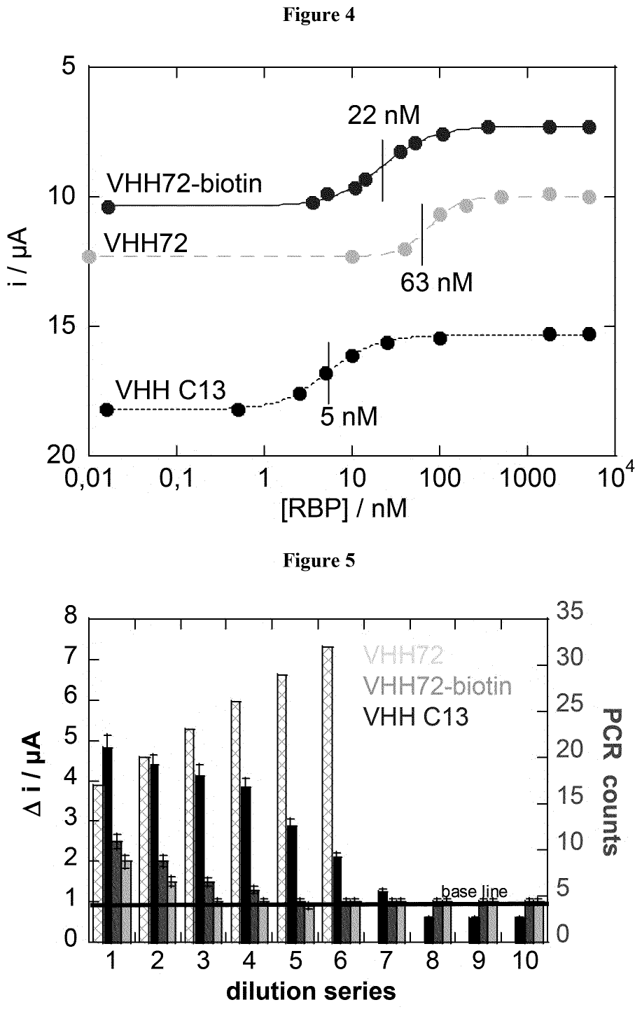

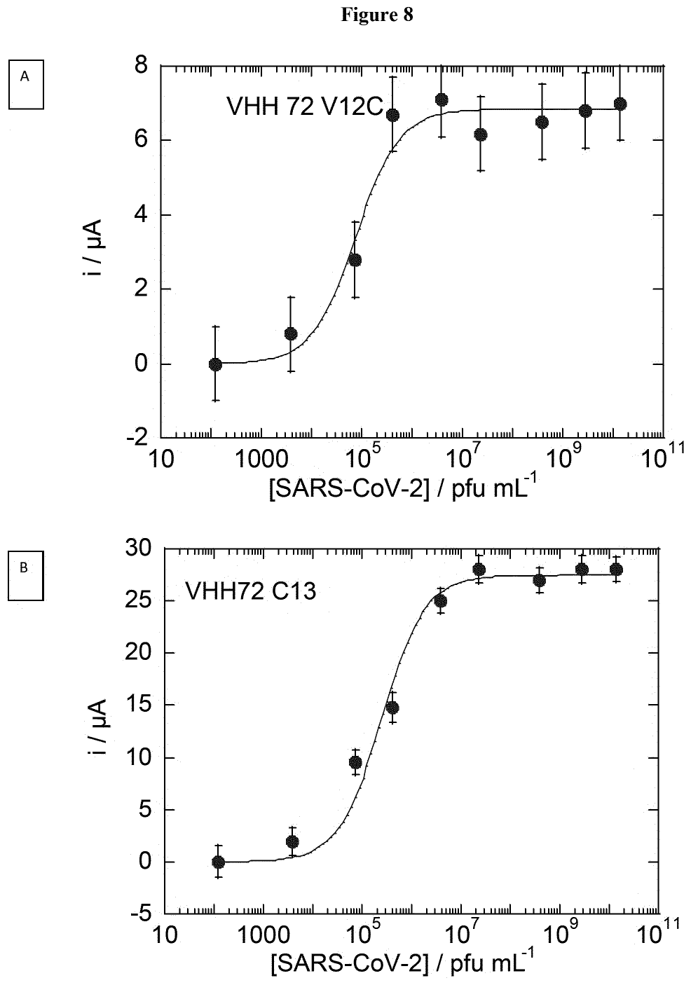

Then, the capacity of each final electrode of specifically binding SARS-CoV-2 receptor binding protein (RBP) and cultured SARS-CoV-2 virus samples was measured by establishing a calibration curve of DPV according to different concentration of SARS-CoV-2 RBP (dilution of stock solution in RBP in PBS (0.1M) and virus samples (dilution of stock solution in SARS-CoV-2 cultured virus in PBS (0.1M)). Incubation for 10 min with the lowest concentration of RBP of cultured virus was started, a DPV signal recorded, and next higher concertation of RBP added. The solution used of the measure is Ferrocenmethanol (FcMeOH, 1 mM) in PBS (0.1M). DPV Parameters are as follows: equilibrium time 3s; Estep=0.01V; Epuls=0.06V; t=0.02s; scan rate: 0.06V/s. The result is in FIG. 4 for RBP and FIGS. 8 (A) and (B) for SARS-CoV-2 virus samples. As can be seen, the mutated nanobody of the present invention VHH C13 binds rapidly at concentrations of 4 to 5 nM of SARS-CoV-2 RBP, whereas for the other electrodes the binding occurs later and at concentrations 4 times and 12 times higher. Similar results were obtained with the SARS-CoV-2 virus samples (see FIGS. 8 (A) and (B)). Therefore, the mutation of the SARS-CoV-2 nanobody at a specific place in its sequence, particularly in position 13, by a cysteine makes it possible to obtain a very sensitive and fast-reading electrode that meets the specifications of a device.

Different diabodies directed to the SARS-CoV-2 RBP were tested instead of nanobodies, equivalents results have been obtained with binding affinity of 0.28 nM, 0.082 nM and 0.018 nM.

These diabodies, as well as, the mutated nanobody of the present invention VHH C13 have been tested on SARS-CoV-2 variants, notably UK, South African and Delta variant. Similar results have been obtained.

### Example 3: Comparison the Mutated Gold Electrode of the Present Invention to RT-PCR Via Dilution Testing

Once the efficacy and sensitivity of the sensing electrode of the present invention was established for SARS-CoV-2, it was compared to the gold standard for the diagnosis of SARS-CoV-2 which is PCR.

Nasopharyngeal specimens were collected from patients in Viral Transport Medium (VTM/UTM) (Yocon®). A strongly positive sample (PCT 18 counts) was 10-fold serially diluted in negative samples.

Extraction with the MGI Easy Nucleic Acid Extraction Kit on the MGISP-960 Automated Sample Preparation System.

RT-PCR with TaqPath™ COVID-19 Combo Kit (Multiplex real-time RT-PCR test intended for the presumptive qualitative detection of nucleic acid from SARS-CoV-2 by Thermofischer®) on the QuantStudio 5 Real-Time PCR System.

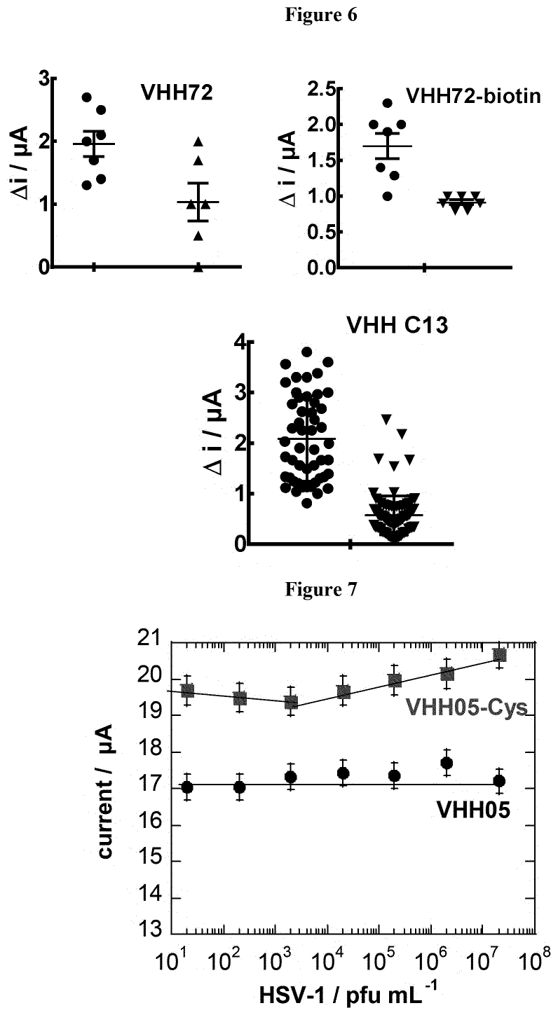

The three gold electrodes of example 2 were tested: the non-mutated SARS-CoV-2 nanobody electrode, the biotin mutated SARS-CoV-2 nanobody electrode and the cysteine mutated SARS-Cov-2 nanobody (VHH C13) electrode of the present invention. The result is in FIG. 6. As demonstrated, the RT-PCR detects SARS-CoV-2 virus until the 5^{th} dilution, non-mutated VHH 72 detects SARS-Cov-2 virus until the 2^{nd} dilution, VHH-biotin detects SARS-Cov-2 virus until the 4^{th} dilution and VHH-72-Cys (VHH C13) of the present invention detects SARS-CoV-2 virus until the 7^{th} dilution. In consequence, the mutated SARS-CoV-2 nanobody of the present invention allows the detection of the SARS-CoV-2 virus at very low concentrations, in a specific way and allows to go beyond the limits of the gold standard diagnostic method by detecting the virus where PCR is no longer able to do so. In consequence, the mutated SARS-Cov-2 nanobody of the present invention can become the new gold standard diagnostic of viruses and particularly SARS-Cov-2 virus.

### Example 4: Clinical Trial of the Mutated SARS-Cov-2 Nanobody of the Present Invention Compared to the PCR Gold Standard

The clinical trial was conducted on the basis of the following protocol.

TABLE 2
study design of the rapid detection of covid-19 by portable and connected
biosensor according to the present invention

| | |
|---|---|
| EXPERIMENTAL | This is a proof-of-concept biological diagnostic study carried out |
| PLAN | on the first nasopharyngeal swab collected from patients admitted |
| | to Lille University Hospital (emergency, hospitalization, |
| | resuscitation) for suspected COVID-19 infection. Samples was |
| | collected systematically during the classical diagnostic |
| | management of covid-19 by first PCR. PCR was performed |
| | according to the usual management. The analysis with a biosensor |
| | according to the invention was performed on the same first |
| | specimen as the PCR. The patient received the usual diagnostic |
| | and therapeutic management. |
| | Biosensor analyses were ideally performed at the same time as |
| | PCR on fresh samples. |
| | Investigators and research associates collected data to establish the |
| | diagnosis. Statistical analyses were performed when the 200 |
| | defined diagnoses (100 positive and 100 negative) based on the |
| | gold standard of medical expertise were obtained. |
| | This is a research mentioned in 3º of article L. 1121-1 of the |
| | public health code because there is no contact by essence with the |
| | in vitro diagnostic medical device, nor any change in the |
| | diagnostic or therapeutic conduct. |
| | There is no risk or constraint associated with the realization of this |
| | biological proof of concept. |
| | The sampling of the elements was carried out within the |
| | framework of the care, for the specific needs of research there is |
| | only: |
| | An in vitro diagnosis of the samples by the biosensor in parallel |
| | with these samples. |
| | A prospective collection of medical data (without additional |
| | constraints). |
| | Measures taken to minimize bias: |
| | Consecutive recruitment of biological samples in the event of a |
| | continuing epidemic. |
| | Blind reading of PCR, biosensor diagnoses and patient |
| | characteristics |
| OBJECTIVES | To study the concordance between the diagnosis made by a |
| | biosensor according to the invention and the diagnosis made by |
| | PCR based on nasopharyngeal swabs taken at the patient's |
| | admission. |
| EVALUATION | Cohen's Kappa Coefficient for concordance for the diagnosis |
| CRITERIA | of CoV-2-SARS between PCR and a biosensor according to the |
| | invention based on nasopharyngeal swabs taken at patient |
| | admission. |
| | The analyses will take into account the confirmed (at the outset or |
| | after repeated examinations) and probable diagnoses made by the |
| | medical team independently of the result of the biosensor analysis. |
| | Doubtful atypical cases (no complete diagnostic approach) will be |
| | excluded before analysis of this first validation study. |
| CRITERIA OF | Male or female or child without age limit |
| INCLUSION | Admitted to a Reference Health Establishment (RHS) in an |
| | emergency unit, hospitalization or intensive care unit for suspicion |
| | of SARS-COV-2 infection, regardless of clinical presentation and |
| | degree of severity. |
| | Patient to be diagnosed by PCR test on nasopharyngeal swab. |
| | Social insured |
| CRITERIA OF | Atypical or suspicious cases without a final diagnosis of COVID- |
| NON-INCLUSION | 19 positive or negative |
| | Refusal of the patient to participate (collection of information, and |
| | second use of the sample, collection of saliva by spitting) |
| | Pregnant and breastfeeding women |
| | Protected Majors |
| NUMBER OF | 200 patients: 100 with a positive diagnosis of SARS-COV-2 and |
| PATIENTS | 100 with a negative diagnosis of SARS-COV-2 defined by the gold |
| | standard by the medical team |
| STATISTICAL | Analysis method and strategy |
| ANALYSES | The statistical analyses will be carried out using SAS software |
| | (version 9.4 or higher). All the statistical tests will be bilateral with a |
| | first species risk of 5%. The quantitative variables will be described |
| | by the mean and standard deviation in case of Gaussian distribution, |
| | or by the median and interquartile (i.e. 25th and 75th percentiles) in |
| | the opposite case. The normality of the distributions will be assessed |
| | graphically by histograms and by the Shapiro-Wilk test. The |
| | qualitative variables will be described by the numbers and |
| | percentages of each modality. |
| | Concordance between PCR and biosensor |
| | To meet secondary objective 1, Cohen's Kappa coefficient between |
| | the diagnosis of CoV-2 SARS made by the biosensor device and the |
| | PCR will be calculated as well as its bilateral 95% confidence |
| | interval. A Kappa value >0.8 will be considered excellent, and a |
| | value between 0.6 and 0.8 will be considered good. The percentage |
| | of agreement will be reported with its bilateral 95% confidence |
| | interval and disagreements will be discussed in comparison to |
| | standard gold. |

Results of the clinical trial are in FIG. 6. As can be seen, the mutated SARS-CoV-2 nanobody (VHH C13) of the present invention allows to have 95% of concordance with PCR results for negative samples, only 5 PCR negative sample which have been detected positive with the mutated SARS-CoV-2 nanobody of the present invention. Regarding PCR positive samples, 99% of concordance is demonstrated, only 1 PCR positive sample which has been detected negative with the mutated SARS-Cov-2 nanobody of the present invention.

### Example 5: Application of the Mutated Nanobody of the Present Invention to HSV-1

Example of HSV-1 nanobody: VHH 05 cys

The sequence of VHH 05 cys is as follows:

(SEQ ID NO: 3)
QVQLVESGGGLVCPGGSLTLSCAASGFSFSTTTMKWVRQAPGKGLERVS
FINRDSTFTQYADSVKGRFTISRDNAKNTLYLQMSSLKPEDTAVYYCAT
ASRITEGADFRGQGTQVTVSSGSHHHHHH

The nanobody contains a C-terminal polyhistidine tag in order to facilitate the purification.

Before the grafting of HSV-1 nanobodies of example 2 on gold electrodes, the gold interfaces were cleaned following the procedure hereafter: Electrodes were irradiated with UV-OZONE for 5 min. Then washed with mQ water dried with a dry air. In the second time, electrodes were cleaned electrochemically with 0.5M H₂SO₄ solution. For this, connect the electrodes to the potentiostat (Palmsens, Sensit), deposit H₂SO₄ on the electrodes and start the cleaning method (Table.1). When the method is finished, rinse electrodes with mQ water and then dry with a dry air. Once cleaned, 2 types of gold electrodes were produced using 2 different nanobodies:
1. Gold electrode grafting with a mutated nanobody of the present invention: VHH 05 cys A solution at 100 μg/mL in PBS 1× of HSV-1 nanobody VHH 05 cys is dropped on electrodes and kept at 4° C. overnight under humid atmosphere. Surfaces were washed, dried and kept at 4° C. until use.
2. Gold electrode grafting with the non-mutated nanobody (VHH 05) using EDC/NHS chemistry on L-Cysteine modified interfaces. A solution L-Cysteine (SigmaAldrich, France) at 2 mM on PBS 1× were dropped on electrodes and kept at 4° C. overnight under humid atmosphere. Electrodes were washed, dried and a solution of NHS/EDC (SigmaAldrich, France) at 15 mM was dropped on surfaces (2h at 4° C.) after 2 hours electrodes were washed, dried and a solution at 100 μg/mL of HSV-1 nanobody VHH 05 was incubated overnight at 4° C. under humid atmosphere. Surfaces were washed, dried and kept at 4° C. until use.

After the grafting of each gold electrode, differential pulse voltammograms (DPV) were measured for each final electrode. The solution used of the measure is Ferrocenmethanol (FcMeOH, 1 mM) in PBS (0.1M). DPV Parameters are as follows: equilibrium time 3s; Estep=0.01V; Epuls=0.06V; t=0.02s; scan rate: 0.06V/s. Then, the capacity of each final electrode of specifically binding HSV-1 virus was measured by establishing a calibration curve of DPV according to different concentration of HSV-1 virus (dilution of stock solution of virus 10⁷ pfu/mL) in PBS (0.1M). Incubation for 10 min with the lowest concertation of HSV-1 virus was started, a DPV signal recorded, and next higher concentration of HSV-1 virus added. The solution used of the measure is Ferrocenmethanol (FcMeOH, 1 mM) in PBS (0.1M). DPV Parameters are as follows: equilibrium time 3 s; Estep=0.01V; Epuls=0.06V; t=0.02s; scan rate:0.06V/s.

The results are shown in FIG. 7. As can be seen, the mutated nanobody of the present invention VHH 05 cys binds rapidly to HSV-1 from 10³ pfu/mL, for the other electrodes no viral binding is observed. Therefore, the mutation of the HSV-1 nanobody at a specific position in its sequence, particularly at position 13, by a cysteine makes it possible to obtain a very sensitive and fast-reading electrode that meets the specifications of a device.

1. A mutated nanobody which binds to a target wherein an amino acid present in a loop of an FR1 framework region of the mutated nanobody is mutated to cysteine.

2. The mutated nanobody of claim 1, wherein the amino acid at position 12, 13, 14 or 15, is mutated to cysteine.

3. The mutated nanobody of claim 1, wherein the amino acid at position 13 is mutated to cysteine.

4. The mutated nanobody of claim 1, wherein the target is selected from the group consisting of viral proteins, protein biomarkers, bacteria proteins, membrane proteins, protozoa proteins, fungi proteins, and prion proteins.

5. The mutated nanobody of claim 4, wherein the target originates from infectious agents.

6. The mutated nanobody of claim 4, wherein the target is a viral protein.

7. The mutated nanobody of claim 1, wherein the mutated nanobody is attached to a surface.

8. The mutated nanobody of claim 7, wherein the surface is a gold surface or a surface selected from the group consisting of graphene, reduced graphene oxide and its derivatives, or a metal surface other than gold such as carbon, platinum, nickel, copper, and silver.

9. The mutated nanobody of claim 7, wherein the surface is a working electrode of a biosensor device.

10. The mutated nanobody of claim 7, wherein the mutated nanobody is attached to the surface with a short linker.

11. A biosensor comprising the mutated nanobody and surface of claim 7.

12. A method for detecting a target in a sample, the method comprising:
a. providing the biosensor of claim 11,
b. contacting the biosensor with the sample,
c. measuring a response at the biosensor surface, and
d. determining the presence or the absence of the target based on the response measured in measuring step c.

13. The method of claim 12, wherein the sample is selected from the group consisting of nasal or mouth swab, spit, and blood.

14. The mutated nanobody of claim 6, wherein the viral protein is a SARS virus protein, a SARS-CoV-2 virus protein, or a SARS-CoV-2 receptor binding protein (RBP).

15. The mutated nanobody of claim 8, wherein the surface is gold or graphene.

16. The mutated nanobody of claim 10, wherein the short linker is a pyrene-based linker or PEG-aryl radical.
